(19) United States
(12) Patent Application Publication
(10) Pub. No.: US 2026/0345653 A1
(43) Pub. Date: Nov. 5, 2026

(54) Composition for the Prevention or Treatment of Self-Injurious Behavior Comprising Ginsenoside Re

(71) Applicant: KNU-INDUSTRY COOPERATION FOUNDATION, Chuncheon-si (KR)

(72) Inventors: Hyoung Chun KIM, Chuncheon-si (KR); Eun Joo SHIN, Chuncheon-si (KR); Sung Kwon KO, Sejong-si (KR)

(73) Assignee: KNU-INDUSTRY COOPERATION FOUNDATION, Chuncheon-si (KR)

(21) Appl. No.: 19/431,178

(22) Filed: Dec. 23, 2025

(30) Foreign Application Priority Data
Dec. 24, 2024 (KR) 10-2024-0195799

(51) Int. Cl. A61K 31/704; A61P 25/00

(52) U.S. Cl. CPC A61K 31/704; A61P 25/00

(57) ABSTRACT

The present invention relates to a composition for the prevention or treatment of self-injurious behavior comprising ginsenoside Re. Specifically, the invention confirms that changes in endogenous antioxidant systems are associated with self-injurious behavior induced by Bay k-8644, and that ginsenoside Re exhibits beneficial effects on these changes. Ginsenoside Re (GRe) restores endogenous antioxidant systems by inhibiting the PKCδ signaling pathway, thereby suppressing self-injurious behavior. Accordingly, the present invention provides a composition for the prevention or treatment of self-injurious behavior comprising ginsenoside Re.

### CROSS REFERENCE TO RELATED APPLICATION

The present application claims priority to Korean Patent Application No. 10-2024-0195799, filed on Dec. 24, 2024, the entire contents of which is incorporated herein for all purposes by this reference.

### BACKGROUND OF THE INVENTION

### 1. Field of the Invention

The present invention relates to a composition for the prevention or treatment of self-injurious behavior comprising ginsenoside Re. More specifically, the present invention confirms whether changes in the endogenous antioxidant system are associated with self-injurious behavior induced by Bay k-8644, and demonstrates that ginsenoside Re exhibits therapeutic effects on such changes. The invention further relates to a composition for the prevention or treatment of self-injurious behavior, wherein GRe suppresses the PKCδ signaling pathway, thereby restoring the endogenous antioxidant system and inhibiting self-injurious behavior.

### 2. Description of the Related Art

Ginseng is classified as an adaptogen, a substance that enhances the body's resistance to harmful physical, chemical, and biological factors and helps restore homeostasis regardless of the direction of abnormal physiological functions. Numerous studies have been conducted regarding the beneficial effects of ginseng.

Ginsenoside Re (GRe) is one of the major ginsenosides contained in ginseng and is known to exert beneficial effects in various neuropsychotoxic and neurodegenerative models. Several studies have suggested that the neuroprotective effects of GRe are associated with its antioxidant activity and its ability to maintain glutathione homeostasis.

Meanwhile, non-suicidal self-injury (NSSI) is defined as behavior that involves cutting oneself with a knife, burning oneself, or severely scratching the skin without suicidal intent. Epidemiological studies indicate that NSSI primarily appears during early to mid-adolescence, although it may occasionally persist into adulthood. NSSI is often a maladaptive behavior in response to emotional problems, and thus comorbid psychiatric disorders, experiences of bullying, neglect, or abuse have been identified as important risk factors.

Neurobiological studies have shown that adolescents who engage in NSSI exhibit abnormal stress-processing mechanisms in the brain, along with increased pain thresholds (Plener et al., 2018). In addition, multiple studies have indicated that NSSI during adolescence is associated with psychiatric disorders, substance abuse, and suicide attempts in adulthood (Liu et al., 2019; Raffagnato et al., 2022; Yan et al., 2023). Animal models for investigating the pathophysiology of NSSI include primates or carnivores that spontaneously exhibit self-injurious behavior, administration of drugs (e.g., pemoline, caffeine, methamphetamine) or neurotoxins (6-hydroxydopamine, Bay k-8644) that induce self-injury, and exposure to adverse environments in early postnatal periods (Devine, 2019; Kita et al., 2000; Novak & Meyer, 2021).

L-type voltage-gated calcium channels (VGCCs) play an important role in regulating the excitability of neurons. Numerous studies have reported that central administration of Bay k-8644, an L-type VGCC activator, can induce seizures accompanied by neuronal cell death. In addition, administration of Bay k-8644 has been shown to induce various forms of self-injurious behavior in mice, including self-biting, and such behaviors are particularly prominent in 3-5-week-old mice, corresponding to early adolescence in humans. Therefore, Bay k-8644 administration has been proposed as a valid model of non-suicidal self-injury (NSSI). Moreover, nifedipine, an L-type VGCC inhibitor, has been reported to exert inhibitory effects in several animal models of self-injurious behavior, and clinical studies have also shown that L-type VGCC inhibitors suppress self-injurious behavior associated with psychiatric disorders, suggesting a potential role of L-type VGCCs in self-injurious behavior.

Functional MRI studies and other neuroimaging investigations conducted in patients with NSSI have indicated that abnormalities in the striatum and brain regions forming neural circuits with the striatum play an important role in the pathophysiological mechanisms of NSSI. Similarly, animal studies have suggested that aberrant changes in the striatum are associated with NSSI. Although the molecular mechanism underlying NSSI is not yet well understood, self-injurious behavior induced by methamphetamine has been reported to be accompanied by oxidative stress in the striatum, and this behavior was suppressed by the free radical scavenger α-phenyl-N-tert-butyl nitrone. Likewise, clinical studies have shown that N-acetylcysteine, a glutathione precursor, is effective in managing NSSI and self-injurious behaviors associated with various psychiatric disorders, suggesting that oxidative stress is involved in the molecular pathology of NSSI. However, it has not yet been reported whether oxidative stress is involved in Bay k-8644-induced self-injurious behavior.

Against this technical background, the present inventors analyzed the effect of ginsenoside Re on Bay k-8644-induced self-injurious behavior and evaluated its potential for preventing or treating such behavior, thereby completing the present invention.

### SUMMARY OF THE INVENTION

The present invention is directed to providing a composition for the prevention or treatment of self-injurious behavior comprising ginsenoside Re.

To achieve the above objective, the present invention provides a composition for the prevention or treatment of self-injurious behavior comprising ginsenoside Re.

In one embodiment, the ginsenoside Re may be administered once daily at a dose of 10 to 50 mg/kg.

In one embodiment, the self-injurious behavior may be non-suicidal self-injury.

In one embodiment, the ginsenoside Re may alleviate or suppress self-injurious behavior by inhibiting PKCδ (protein kinase C δ) phosphorylation observed in such behavior, inhibiting changes in glutathione and NRF2 (nuclear factor erythroid-2-related factor 2) homeostasis, and inhibiting the decrease in antioxidant enzyme activity.

Through the present invention, it was confirmed that in mice, Bay k-8644-induced self-injurious behavior was accompanied by abnormalities in the endogenous antioxidant system and a reduction in Nrf2 activity within the striatum, along with an increase in PKCδ phosphorylation. Pretreatment with GRe inhibited the Bay k-8644-induced expression of p-PKCδ, and suppressed the decrease in Nrf2 activity, the resulting reduction in the expression of GCL enzymes, and the decrease in the GSH/GSSG ratio. GRe further significantly inhibited the Bay k-8644-induced reduction in antioxidant enzyme activity as well as the self-injurious behavior itself. PKCδ gene deficiency also significantly suppressed these changes, indicating a central role of PKCδ in Bay k-8644-induced self-injurious behavior and the associated alterations in the endogenous antioxidant system. Moreover, because GRe did not produce additional inhibitory effects in PKCδ KO mice, it is suggested that the inhibitory action on PKCδ is involved in the effects of GRe. In conclusion, these results indicate that GRe can mitigate Bay k-8644-induced self-injurious behavior by restoring Nrf2 activity and the endogenous antioxidant system through a PKCδ-inhibitory mechanism.

### BRIEF DESCRIPTION OF THE DRAWINGS

FIG. 1A depicts an experimental design that examines time-dependent changes in the endogenous antioxidant system following Bay k-8644-induced self-injurious behavior. FIG. 1B depicts an experimental design that examines the effect of ginsenoside Re (GRe) on Bay k-8644-induced self-injurious behavior.

GRe was administered orally once daily at a dose of 20 mg/kg for 5 days. #, analysis of self-injurious behavior.

FIG. 2. Time-dependent changes in antioxidant enzyme activities and protein kinase Cô (PKCδ) phosphorylation in the striatal tissue of mice following Bay k-8644-induced self-injurious behavior. GPx, glutathione peroxidase. Each value represents mean±SEM of 6-8 mice. *P<0.05, **P<0.01 vs. 0 h after Bay k-8644 (analyzed by one-way ANOVA with Tukey's multiple pairwise comparisons as post hoc analysis).

FIG. 3. Time-dependent changes in reduced glutathione (GSH)/oxidized glutathione (GSSG) ratio, glutathione reductase (GR) activity, and Nrf2 activity in the striatal tissue of mice following Bay k-8644-induced self-injurious behavior. Nrf2 activity was assessed by nuclear translocation and DNA-binding activity. Each value represents mean±SEM of 6-8 mice. *P<0.05, **P<0.01 vs. 0 h after Bay k-8644 (analyzed by one-way ANOVA with Tukey's multiple pairwise comparisons as post hoc analysis).

FIG. 4. Effect of GRe on the expression of p-PKCδ induced by Bay k-8644 in the striatum of mice. Sal, saline; Veh, vehicle. Each value represents mean±SEM of 6 mice. **P<0.01 vs. Sal+Veh. ##P<0.01 vs. Sal+Bay k-8644 (analyzed by two-way ANOVA with Tukey's multiple pairwise comparisons as post hoc analysis).

FIG. 5. Effects of GRe and PKCδ gene deficiency (KO) on the decrease in Nrf2 activity, mRNA expression of glutamate-cysteine ligase catalytic subunit (GCLc) and modulatory subunit (GCLm), GSH/GSSG ratio, and GR activity induced by Bay k-8644 in the striatum of mice. WT, wild-type mice; Sal, saline; Veh, vehicle. Each value represents mean±SEM of 6-8 mice. *P<0.05, **P<0.01 vs. WT/Sal+Veh. #P<0.05, ##P<0.01 vs. WT/Sal+Bay k-8644 (analyzed by three-way ANOVA with Tukey's multiple pairwise comparisons as post hoc analysis).

FIG. 6. Effects of GRe and PKCδ gene deficiency (KO) on the decrease in GPx activity and GPx-1 expression induced by Bay k-8644 in the striatum of mice. WT, wild-type mice; Sal, saline; Veh, vehicle. Each value represents mean±SEM of 6-8 mice. *P<0.05, **P<0.01 vs. WT/Sal+Veh. #P<0.05, ##P<0.01 vs. WT/Sal+Bay k-8644 (analyzed by three-way ANOVA with Tukey's multiple pairwise comparisons as post hoc analysis).

FIG. 7. Effects of GRe and PKCδ gene deficiency (KO) on Bay k-8644-induced self-biting behavior, associated tissue damage (self-injurious behavior), and dystonia in mice. WT, wild-type mice; Sal, saline. Each value represents mean±SEM of 8-9 mice. *P<0.05, **P<0.01 vs. WT/Sal+Veh. #P<0.05, ##P<0.01 vs. WT/Sal+Bay k-8644 [Panels A and D analyzed by two-way ANOVA; panel C analyzed by two-way repeated measures ANOVA, followed by Tukey's multiple pairwise comparisons. Panel B analyzed by Chi-square test].

### DETAILED DESCRIPTION

Unless otherwise defined, all technical and scientific terms used in this specification have the same meaning as commonly understood by those skilled in the art to which the present invention pertains. In general, the nomenclature used in this specification is well known and commonly employed in the art.

The present invention relates to a composition for the prevention or treatment of self-injurious behavior comprising ginsenoside Re. The invention further relates to a method for preventing or treating self-injurious behavior by administering ginsenoside Re to a subject.

The self-injurious behavior may be non-suicidal self-injury. Non-suicidal self-injury (NSSI) is defined as behavior involving cutting oneself with a knife, burning oneself, or severely scratching the skin without suicidal intent.

Ginsenosides, which are saponins contained in ginseng and glycosides of ginseng (Ginseng), are classified into three groups depending on the structure of their aglycone: protopanaxadiol-type (PPD-type) ginsenosides, protopanaxatriol-type (PPT-type) ginsenosides, and oleanolic acid-type ginsenosides. To date, more than approximately 180 ginsenoside species have reportedly been isolated. Among them, ginsenoside Re is a protopanaxatriol-type (PPT-type) ginsenoside belonging to the dammarane-type saponins.

The ginsenoside Re may alleviate or inhibit self-injurious behavior by inhibiting PKCδ (protein kinase C δ) phosphorylation observed in such behavior, suppressing changes in glutathione and NRF2 (nuclear factor erythroid-2-related factor 2) homeostasis, and preventing the reduction of antioxidant enzyme activity.

Through previous studies, it has been confirmed that the antioxidant and glutathione homeostasis-maintaining effects of GRe in excitotoxicity models, serotonin syndrome models, and dopaminergic neurotoxicity models are associated with its inhibitory action on protein kinase Cδ (PKCδ).

Through the present invention, it was examined whether changes in the endogenous antioxidant system are involved in Bay k-8644-induced self-injurious behavior, and whether GRe exerts therapeutic effects on such changes. In addition, the mechanism of action of GRe was investigated. The results of this study demonstrated that GRe suppresses the PKCδ signaling pathway, thereby restoring the endogenous antioxidant system and inhibiting Bay k-8644-induced self-injurious behavior.

The present invention relates to a pharmaceutical composition for the prevention or treatment of self-injurious behavior comprising ginsenoside Re.

The term “prevention,” as used herein, refers to any act of suppressing or delaying the symptoms of self-injurious behavior through administration of the composition according to the present invention. The term “treatment or improvement” refers to any act in which the symptoms of self-injurious behavior are improved or beneficially altered.

The pharmaceutical composition may be formulated into a unit dosage form or filled into a multi-dose container using pharmaceutically acceptable carriers and/or excipients, according to methods that can be readily practiced by a person skilled in the art. The formulation may be in the form of a solution, suspension, or emulsion in an oil or aqueous medium, or in the form of an extract, powder, granule, tablet, capsule, or gel (e.g., hydrogel), and may additionally include dispersing agents or stabilizers.

The pharmaceutical composition may further comprise, in addition to the active ingredient, pharmaceutically acceptable adjuvants such as preservatives, stabilizers, hydrants or emulsifying aids, salts and/or buffers for osmotic pressure control, as well as other substances useful for improving formulation properties. The composition may be formulated into various oral or parenteral dosage forms according to conventional methods. Examples of oral dosage forms include tablets, pills, hard and soft capsules, liquids, suspensions, emulsions, syrups, powders, troches, fine granules, granules, and pellets. These formulations may additionally contain, aside from the active ingredient, surfactants, diluents (e.g., lactose, dextrose, sucrose, mannitol, sorbitol, cellulose, and glycine), and lubricants (e.g., silica, talc, stearic acid and its magnesium or calcium salts, and polyethylene glycol). Tablets may further contain binders such as magnesium aluminum silicate, starch paste, gelatin, tragacanth, methylcellulose, sodium carboxymethylcellulose, and polyvinylpyrrolidone, and may additionally contain, when necessary, disintegrants such as starch, agar, alginic acid or sodium alginate, absorbents, colorants, flavoring agents, and sweeteners. Tablets may be manufactured by conventional mixing, granulation, or coating methods.

Pharmaceutically acceptable carriers may include, but are not limited to, lactose, glucose, sucrose, sorbitol, mannitol, starch, acacia, gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil, which are typically used during formulation. In addition to these components, lubricants, wetting agents, sweeteners, flavoring agents, emulsifiers, suspending agents, and preservatives may be further included.

The pharmaceutical composition may be administered orally or parenterally and may be used in the form of a conventional pharmaceutical preparation. That is, the pharmaceutical composition of the present invention may be administered in various oral and parenteral dosage forms in actual clinical use, and when formulated, it is typically prepared using diluents or excipients such as fillers, extenders, binders, wetting agents, disintegrants, and surfactants commonly used in the field. Solid formulations for oral administration include tablets, pills, powders, granules, and capsules, and such solid formulations are prepared by mixing at least one excipient—such as starch, calcium carbonate, sucrose or lactose, or gelatin—with a herbal extract or herbal fermentation product. In addition to simple excipients, lubricants such as magnesium stearate and talc may also be used. Liquid formulations for oral administration include suspensions, solutions, emulsions, and syrups, and may additionally include various excipients such as wetting agents, sweeteners, flavoring agents, and preservatives, in addition to simple diluents commonly used, such as water or liquid paraffin. Parenteral dosage forms include sterile aqueous solutions, non-aqueous solvents, suspensions, emulsions, freeze-dried preparations, and suppositories. Examples of non-aqueous solvents and suspending agents include propylene glycol, polyethylene glycol, vegetable oils such as olive oil, and injectable esters such as ethyl oleate. Examples of suppository bases include Witepsol, macrogol, Tween 61, cocoa butter, lauric oil, glycerol, and gelatin.

The concentration of the active ingredient included in the composition may be determined by considering therapeutic objectives, the patient's condition, and the required duration of treatment, and is not limited to any particular range. The pharmaceutical composition of the present invention is administered in a pharmaceutically effective amount. As used herein, the term “pharmaceutically effective amount” refers to an amount sufficient to treat a disease with a reasonable benefit-to-risk ratio applicable to medical treatment, and the effective dosage level may vary depending on factors well known in the medical field, such as the type and severity of the patient's condition, the activity of the drug, sensitivity to the drug, dosing time, route of administration, elimination rate, duration of treatment, and concomitant medications. The pharmaceutical composition of the present invention may be administered as a monotherapy or in combination with therapeutic agents for diseases induced by other contaminants, and may be administered simultaneously, separately, or sequentially with conventional therapeutic agents, either as a single dose or multiple doses. Considering all these factors, it is important to administer an amount that maximizes the therapeutic effect with minimal side effects, and such determination can be readily made by a person skilled in the art. The effective dose may vary depending on the patient's age, sex, condition, body weight, the degree of absorption, inactivation, or excretion of the active ingredient in the body, the type of disease, and concomitant medications, and may be increased or decreased depending on route of administration, severity, sex, body weight, and age.

The ginsenoside Re may be administered once daily at a dose of 10 to 50 mg/kg.

The present invention also relates to a health functional food composition for the prevention or treatment of self-injurious behavior comprising ginsenoside Re.

The food composition may be in liquid or solid form, including, for example, various types of foods, beverages, gums, teas, multivitamin preparations, and health supplements, and may be used in the form of powders, granules, tablets, capsules, or drinks. In each formulation, components commonly used in the field may be appropriately selected and blended by a person skilled in the art according to the formulation or intended use in addition to the active ingredient, and synergistic effects may occur when combined with other raw materials. There is no particular limitation on the liquid components that may be included in addition to the active ingredient disclosed in this specification, and the composition may further include additional ingredients such as various flavoring agents or natural carbohydrates, similar to conventional beverages. Examples of natural carbohydrates include monosaccharides, disaccharides such as glucose and fructose, polysaccharides such as maltose and sucrose, conventional sugars such as dextrin and cyclodextrin, and sugar alcohols such as xylitol, sorbitol, and erythritol. Natural flavoring agents such as thaumatin and stevia extracts (e.g., rebaudioside A, glycyrrhizin) and synthetic flavoring agents (e.g., saccharin, aspartame) may be advantageously used. The amount of natural carbohydrates may generally be about 1 to 20 g per 100 ml of the composition disclosed herein, and in one aspect about 5 to 12 g. The food composition may also contain, in one aspect, various nutrients, vitamins, minerals (electrolytes), synthetic flavoring agents and natural flavoring agents, colorants, thickeners (such as cheese or chocolate), pectic acid and salts thereof, alginic acid and salts thereof, organic acids, protective colloid thickeners, pH adjusters, stabilizers, preservatives, glycerin, alcohols, and carbonating agents used in carbonated beverages.

The health functional food refers to a food processed to exhibit not only nutritional supplementation but also effective physiological regulatory functions, and may be manufactured in various forms such as tablets, capsules, powders, granules, liquids, or pills to obtain beneficial effects for the prevention or improvement of vaginitis.

The health functional food composition may be manufactured as a food, particularly a functional food. The functional food of the present invention may include ingredients commonly added during food manufacturing, such as proteins, carbohydrates, fats, nutrients, and seasonings. For example, when manufactured as a drink formulation, natural carbohydrates or flavoring agents may be included as additional components in addition to the active ingredient. The natural carbohydrates are preferably monosaccharides (e.g., glucose, fructose), disaccharides (e.g., maltose, sucrose), oligosaccharides, polysaccharides (e.g., dextrin, cyclodextrin), or sugar alcohols (e.g., xylitol, sorbitol, erythritol). The flavoring agent may include natural flavoring agents (e.g., thaumatin, stevia extracts) and synthetic flavoring agents (e.g., saccharin, aspartame).

There is no particular limitation on the type of health functional food. Examples include dairy products, beverages, tea drinks, alcoholic beverages, and multivitamin preparations, and all foods generally considered health foods may be included.

Additionally, the composition may further include various nutrients, vitamins, electrolytes, flavoring agents, colorants, pectic acid and salts thereof, alginic acid and salts thereof, organic acids, protective colloid thickeners, pH adjusters, stabilizers, preservatives, glycerin, alcohols, and carbonating agents used in carbonated beverages.

### Example

The following examples are provided to describe the present invention in further detail. These examples are presented solely for illustrative purposes, and it will be apparent to those skilled in the art that the scope of the present invention is not limited by these examples.

### 1. Materials and Methods

### 1.1 Experimental Animals

All animals were used in accordance with the Guide for the Humane Care and Use of Laboratory Animals of the U.S. National Institutes of Health (NIH) (2015 Edition; grants.nih.gov/grants/olaw/references/PHSPolicyLabAnimals.pdf) and the Guide for the Care and Use of Laboratory Animals of the Institute for Laboratory Animal Research (ILAR) (8th Edition; grants.nih.gov/grants/olaw/Guide-for-the-care-and-use-of-laboratory-animals.pdf). All animal experimental procedures were approved by the Institutional Animal Care and Use Committee (IACUC) of Kangwon National University (#KW-240226-1).

PKCδ gene-deficient (KO) mice (Nature, 416 (6883), 865-869) were generated by obtaining C57BL/6 background PKCδ (±) mice for breeding from Dr. K. I. Nakayama (Department of Molecular Genetics, Medical Institute of Bioregulation, Kyushu University, Fukuoka, Japan). Genotypes were confirmed by PCR using DNA extracted from tail tissue. PKCδ KO mice and wild-type (WT) mice obtained from breeding were used for the experiments. All mice were housed under a 12 h light/dark cycle in a facility with controlled temperature and humidity, with free access to water and food.

### 1.2 Drug Administration

Bay k-8644 (Biomol Research Laboratories, Inc., Plymouth Meeting, PA, USA) was dissolved in sterile physiological saline containing 10% Tween 80. GRe was dissolved in sterile physiological saline.

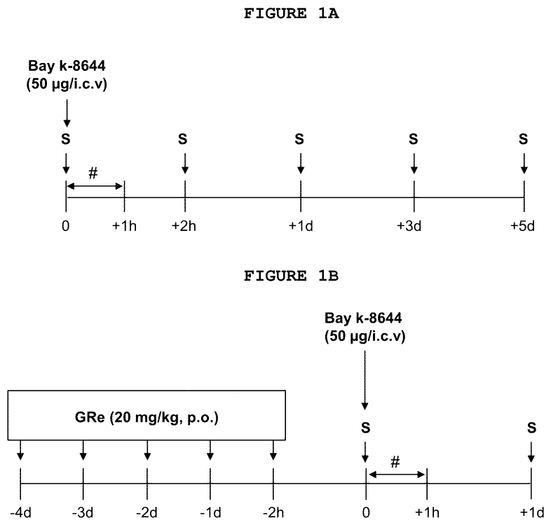

First, the time-dependent changes in the endogenous antioxidant system in the striatum after Bay k-8644 administration were examined. For this purpose, 5-week-old wild-type mice were sacrificed by cervical dislocation at 0 h, 2 h, 1 day, 3 days, and 5 days after intracerebroventricular administration of Bay k-8644 (50 μg/mouse) into the third ventricle, and striatal tissues were isolated. Using separate mice, the effect of GRe on Bay k-8644-induced self-injurious behavior was examined. For this, PKCδ KO mice and WT mice were administered GRe (20 mg/kg, p.o.) once daily for 5 days, and 2 h after the last GRe administration, Bay k-8644 (50 μg/mouse) or vehicle (sterile saline containing 10% Tween 80, 1 L/mouse) was administered intracerebroventricularly. The dose of GRe was determined based on our previous studies (Shin et al., 2014; Tran et al., 2023). Mice were euthanized by cervical dislocation or by cardiac perfusion under urethane anesthesia (1.5 g/kg, i.p.). The experimental design is shown in FIG. 1.

1.3 Analysis of Bay k-8644-Induced Self-Injurious Behavior

Self-biting behavior and tissue damage resulting from self-injury were analyzed for 1 hour following Bay k-8644 administration, based on methods established in previous studies (Dev Neurosci, 24 (4), 322-327).

Self-biting behavior was defined as biting of the body without resulting in actual tissue injury.

Tissue damage resulting from self-injury was defined as biting behavior that produces actual tissue damage or bleeding.

Self-biting behavior was observed for 1 hour in 10-minute intervals, and the number of intervals exhibiting self-biting was expressed as a percentage. Tissue damage resulting from self-injury was expressed as the percentage of mice exhibiting tissue damage in each group.

Additionally, dystonia was assessed. Dystonia was defined as abnormal movements characterized by twisting of the body due to abnormal muscle tone, and was graded based on previously established methods as follows:
Grade 0: Normal movement
Grade 1: Slightly slowed movement
Grade 2: Mild abnormal movement (barely walks unless stimulated, occasional abnormal posture, rare falling)
Grade 3: Moderate abnormal movement (barely walks even if stimulated, frequent abnormal posture, nearly always falls when standing)
Grade 4: Severe abnormal movement (rarely walks, continuously exhibits abnormal posture, almost unable to stand)

Dystonia was also observed for 1 hour in 10-minute intervals, and the dystonia grade observed in each interval was recorded.

### 1.4. Analysis of Glutathione Peroxidase (GPx) Activity

GPx activity was measured as previously reported in Ginsenoside Re rescues methamphetamine-induced oxidative damage, mitochondrial dysfunction, microglial activation, and dopaminergic degeneration by inhibiting the protein kinase Cdelta gene (Mol Neurobiol, 49 (3), 1400-1421) and Ginsenoside Re blocks Bay k-8644-induced neurotoxicity via attenuating mitochondrial dysfunction and PKCdelta activation in the hippocampus of mice: Involvement of antioxidant potential (Food Chem Toxicol, 178, 113869).

Striatal samples (0.3-0.8 mg protein) were incubated for 5 minutes at 25 °C in 50 mM phosphate buffer containing 1.4 IU glutathione reductase, 0.2 mM NADPH, and 2 mM reduced glutathione, after which 0.25 mM cumene hydroperoxide was added to initiate the reaction. ΔOD/min was measured for 5 minutes at 340 nm using a microplate reader (Spectra Max Plus 384, Molecular Devices Inc.). GPx activity was calculated as nmol of NADPH oxidized per minute per mg of protein using the extinction coefficient of NADPH (6.22 Mm⁻¹·cm⁻¹).

### 1.5. Analysis of Glutathione Reductase (GR) Activity

GR activity was measured as previously reported (Eklow, L., Moldeus, P., & Orrenius, S. (1984). Oxidation of glutathione during hydroperoxide metabolism. A study using isolated hepatocytes and the glutathione reductase inhibitor 1,3-bis(2-chloroethyl)-1-nitrosourea. Eur J Biochem, 138 (3), 459-463). A 100 μL sample was added to phosphate buffer (pH 7.8) containing 1 mM oxidized glutathione and 1 mM EDTA, and the reaction was initiated by adding NADPH (final concentration: 0.11 mM). The decrease in NADPH absorbance (ΔOD/min) at 340 nm and 25 °C was measured using a microplate reader (Spectra Max Plus 384, Molecular Devices Inc.). One unit of GR activity was defined as the rate at which 1 μmol of NADPH was oxidized per minute, and activity was calculated from ΔOD/min using the extinction coefficient of NADPH (6.22 mM⁻¹·cm⁻¹).

### 1.6. Measurement of Glutathione Levels

Striatal glutathione levels were measured using the enzymatic recycling method. Striatal tissue was homogenized in phosphate buffer (100 mM, pH 7.5) containing 0.1% Triton X-100, 0.6% sulfosalicylic acid, and 5 mM EDTA. The homogenate was centrifuged at 8,000×g for 10 minutes at 4 °C, and the resulting supernatant was used for analysis. To measure total glutathione, the extracted supernatant was added to a reaction mixture containing glutathione reductase (250 units/mL) and β-NADPH (2 mg in 3 mL phosphate buffer) to reduce oxidized glutathione (GSSG) to reduced glutathione (GSH). The total GSH was reacted with 5,5′-dithio-bis(2-nitrobenzoic acid) (DTNB, 2 mg in 3 mL phosphate buffer) to form the 5-thio-2-nitrobenzoic acid (TNB) chromophore. Absorbance was measured at 412 nm for 2 minutes. The rate of TNB formation was calculated and compared to a standard curve generated using various concentrations of glutathione to determine total glutathione levels. For GSSG measurement, the extracted supernatant was first incubated in phosphate buffer containing 10% 2-vinylpyridine at 20 °C for 1 hour. This reagent forms covalent bonds with GSH, preventing its interaction with glutathione reductase. Excess vinylpyridine was neutralized with triethanolamine to pH 6-7, and the mixture was then reacted with glutathione reductase and β-NADPH. The rate of TNB formation from DTNB was recorded at 412 nm for 2 minutes. GSH concentration was calculated by subtracting twice the GSSG concentration from the total glutathione concentration.

### 1.7. Western Blot Analysis

Striatal tissues were homogenized in lysis buffer (pH 6.8) containing 1% SDS, 5 mM EGTA, 10% glycerol, 5 mM EDTA, phosphatase inhibitor cocktail (Sigma-Aldrich), and protease inhibitor cocktail (Sigma-Aldrich), followed by centrifugation at 12,000×g for 30 minutes. The supernatant was subjected to 10% SDS-PAGE (20-50 μg of protein per lane) and transferred to a polyvinylidene difluoride membrane.

The membrane was blocked with 3% nonfat milk and incubated with primary antibodies overnight at 4 °C. After washing, the membrane was incubated with HRP-conjugated secondary antibodies, and protein bands were visualized using an enhanced chemiluminescence kit (GE Healthcare, Piscataway, NJ, USA). Band intensity was quantified using PhotoCapt MW (version 10.01 for Windows, Vilber Lourmat, Marne la Vallée, France) and normalized to β-actin. The primary antibodies used are listed in Table 1.

TABLE 1

| | | Dilution | | Product |
|---|---|---|---|---|
| Antigen | Species | Used | Company | Number |
| p-PKCδ | Mouse | 1:500 | Santa Cruz Biotechnology | sc-377560 |
| PKCδ | Mouse | 1:1000 | Santa Cruz Biotechnology | sc-8402 |
| GPx-1 | Goat | 1:1000 | R&D Systems, Minneapolis, | AF3798 |
| | | | MN, USA | |
| Nrf2 | Rabbit | 1:1000 | Santa Cruz Biotechnology | sc-13032 |
| Nuclear | Mouse | 1:1000 | Abcam, Cambridge, MA, | ab487 |
| matrix | | | USA | |
| β-actin | Mouse | 1:300000 | Sigma-Aldrich (St. Louis, | A5441 |
| | | | MO, USA) | |

### 1.8. Analysis of Nuclear Translocation of Nrf2

Nuclear fractions of striatal tissue were obtained using the NE-PER Nuclear and Cytoplasmic Extraction Kit (Thermo Scientific, Rockford, IL, USA). Briefly, striatal tissue was placed in the cytoplasmic extraction reagent provided in the kit and homogenized using a Dounce homogenizer. The pellet was then resuspended in the nuclear extraction reagent supplied with the kit and centrifuged at 16,000×g for 10 minutes. The resulting supernatant, representing the nuclear fraction, was subjected to Western blot analysis for Nrf2 to assess its nuclear translocation.

### 1.9. Analysis of Nrf2 DNA-Binding Activity

Nuclear fractions for DNA-binding activity analysis were prepared using a nuclear extraction kit (#40410; Active Motif, Carlsbad, CA, USA). Briefly, tissue was homogenized in the hypotonic buffer provided in the kit and centrifuged at 850×g for 15 minutes. The pellet was resuspended in hypotonic buffer again and centrifuged at 14,000×g for 30 seconds. The resulting pellet constituted the nuclear fraction, which was lysed in complete lysis buffer provided in the kit and used for subsequent analyses. Protein concentrations of nuclear fractions were measured using the BCA protein assay kit (Thermo Scientific). Nrf2 DNA-binding activity was assessed using the TransAM™ Nrf2 kit (#50296, Active Motif, Carlsbad, CA, USA). Briefly, nuclear extracts were added to a 96-well plate coated on the bottom with oligonucleotides containing the ARE consensus binding site (5′-GTCACAGTGACTCAGCAGAATCTG-3′) and incubated at room temperature for 1 hour. After washing each well with the wash buffer supplied in the kit, Nrf2 bound to the ARE consensus site was detected using an Nrf2 antibody. The wells were then incubated with an HRP-conjugated secondary antibody for 1 hour, and absorbance was measured at 450 nm using a microplate reader (Spectra Max Plus 384, Molecular Devices Inc.).

### 1.10. Real-Time PCR

Total RNA from the striatum was extracted using the RNeasy Mini Kit (Qiagen, Valencia, CA, USA), and 2 μg of RNA was converted to cDNA using the RNA to cDNA EcoDry Premix (Takara, San Jose, CA, USA). An equal amount of cDNA from each sample was added to a reaction mixture containing 10 μmol of each primer and SsoAdvanced™ Universal SYBR Green Supermix (Bio-Rad Laboratories, Inc., Hercules, CA, USA), followed by amplification using the CFX96 Touch Real-Time PCR System (Bio-Rad Laboratories, Inc.). The reference gene GAPDH and the target genes were amplified simultaneously on the same plate.

The primers used were as follows:

GCLc:
5′-CTACCACGCAGTCAAGGACC-3′ (forward)
and
5′-CCTCCATTCAGTAACAACTGGAC-3′ (reverse)
GCLm:
5′-GCCACCAGATTTGACTGCCTTTG-3′ (forward)
and
5′-TGCTCTTCACGATGACCGAGTACC-3′ (reverse)
GAPDH:
5′-ACCACAGTCCATGCCATCAC-3′ (forward)
and
5′-TCCACCACCCTGTTGCTGTA-3′ (reverse)

The DNA polymerase was activated at 95 °C for 3 minutes, followed by 40 cycles of denaturation at 95 °C for 10 seconds and annealing/extension at 58-60 °C for 30 seconds. mRNA expression of the target genes was calculated using the 2^{-ΔΔCt} method.

### 1.11. Statistical Analysis

Data were analyzed using IBM SPSS version 26.0 (IBM Corporation). Self-injurious behavior was analyzed using the Chi-square test. Time-dependent changes were evaluated using one-way analysis of variance (ANOVA), whereas the effects of Bay k-8644 and GRe were assessed using two-way ANOVA. The interactive effects of PKCδ knockout, Bay k-8644, and GRe were analyzed using three-way ANOVA. Dystonia scores were analyzed using repeated-measures ANOVA. Post hoc analyses were performed using Tukey's multiple pairwise comparisons or multiple pairwise comparisons using estimated marginal means with Bonferroni adjustment. A p-value <0.05 was considered statistically significant.

### Example 1. Bay k-8644-Induced Self-Injurious Behavior was Accompanied by Reduced Antioxidant Enzyme Activity in the Striatum

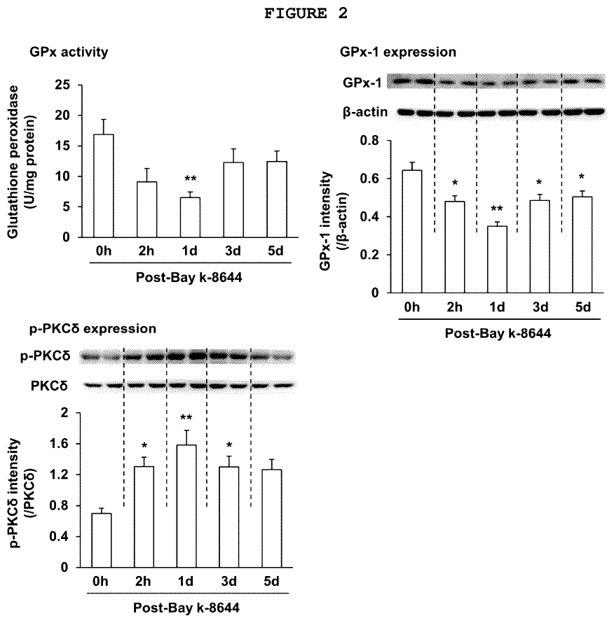

We first examined the time-dependent changes in antioxidant enzyme activity in the striatum following Bay k-8644-induced self-injurious behavior. GPx activity significantly decreased 1 day after Bay k-8644 administration (P<0.01 vs. 0 h). Because the most prominent reduction in GPx activity occurred at 1 day, we further examined the expression of GPx-1, the most abundant GPx isoform in the central nervous system. GPx-1 expression in the striatum was also significantly decreased after Bay k-8644 injection (2 h, 3 d, and 5 d vs. 0 h, P<0.05; 1 d vs. 0 h, P<0.01), consistent with the reduction observed in GPx activity (FIGS. 2A-D). These results indicate that Bay k-8644-induced self-injurious behavior is accompanied by a decrease in antioxidant enzyme activity in the striatum.

### Example 2. Phosphorylation of PKCδ was Increased in the Striatum Following Bay k-8644 Induced Self-Injurious Behavior

Because Bay k-8644-induced self-injurious behavior was accompanied by reduced antioxidant enzyme activity in the striatum, we next examined the phosphorylation of PKCδ, a representative redox-sensitive kinase. Administration of Bay k-8644 significantly increased the expression of phosphorylated PKCδ (p-PKCδ) in the striatum (2 h, 3 d, and 5 d vs. 0 h, P<0.05; 1 d vs. 0 h, P<0.01). The increase in p-PKCδ expression was most prominent 1 day after Bay k-8644 administration, which coincides with the time point at which the most substantial reduction in antioxidant enzyme expression was observed (FIG. 2E). These results indicate that the decrease in antioxidant enzyme activity observed after Bay k-8644-induced self-injurious behavior occurs in parallel with an increase in p-PKCδ expression in the striatum.

### Example 3. Bay k-8644-Induced Self-Injurious Behavior was Accompanied by Disrupted Glutathione Homeostasis and Reduced Nrf2 DNA-Binding Activity in the Striatum

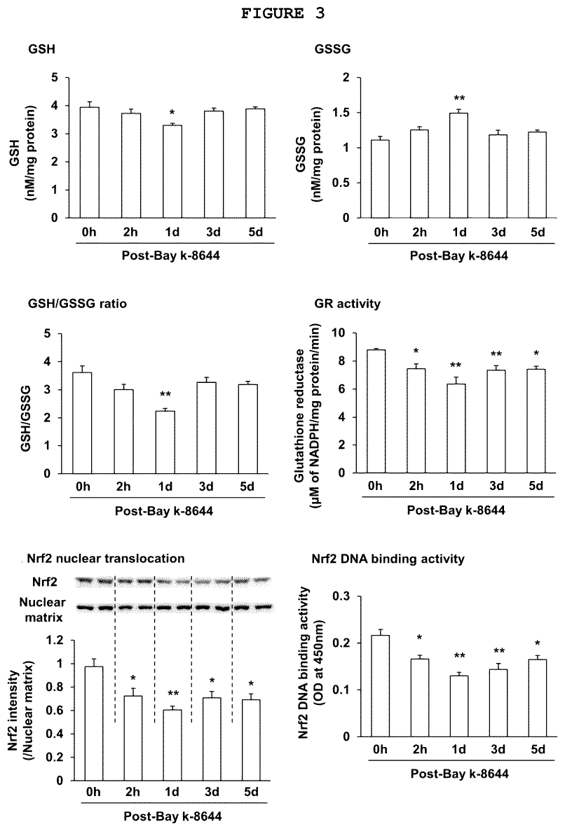

We next assessed glutathione homeostasis by measuring the ratio of reduced to oxidized glutathione (GSH/GSSG), the levels of GSH and GSSG, and the activity of glutathione reductase (GR), the enzyme responsible for reducing GSSG back to GSH. In addition, we examined the DNA-binding activity of Nrf2, a transcription factor that promotes the transcription of glutamate-cysteine ligase (GCL), the rate-limiting enzyme for glutathione synthesis. One day after Bay k-8644 administration, GSH levels (P<0.05 vs. 0 h) and the GSH/GSSG ratio (P<0.01 vs. 0 h) in the striatum were significantly decreased, whereas GSSG levels were significantly increased (P<0.01 vs. 0 h) (FIGS. 3A-C). Consistently, both nuclear translocation and DNA-binding activity of Nrf2, as well as GR activity, were significantly decreased 2 hours after Bay k-8644 administration, and these reductions persisted up to 5 days (GR activity and Nrf2 DNA-binding activity: 2 h and 5 d vs. 0 h, P<0.05; 1 d and 3 d vs. 0 h, P<0.01; Nrf2 nuclear translocation: 2 h, 3 d, and 5 d vs. 0 h, P<0.05; 1 dvs. 0 h, P<0.01) (FIGS. 3D-F). These findings demonstrate that Bay k-8644-induced self-injurious behavior is accompanied by impaired glutathione homeostasis and associated abnormalities in Nrf2 signaling within the striatum.

### Example 4. GRe Inhibited Bay k-8644-Induced Phosphorylation of PKCδ in the Striatum

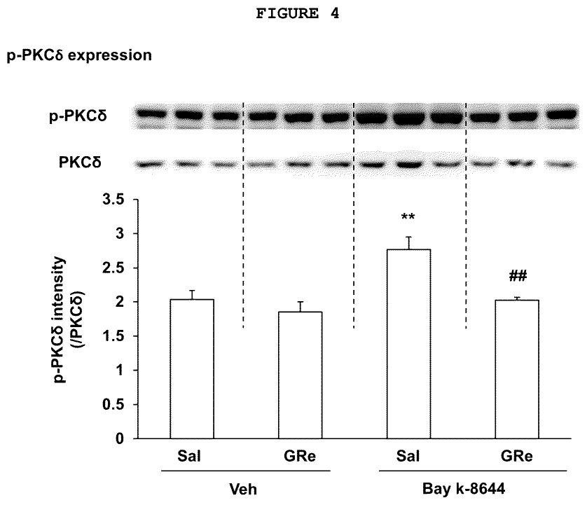

Because the inhibitory effect of GRe on PKCδ has been shown to play an important role in its neuroprotective actions, we examined whether GRe suppresses the increase in p-PKCδ expression induced by Bay k-8644. As the Bay k-8644-induced increase in p-PKCδ expression was most prominent 1 day after administration, the effect of GRe was also evaluated at this time point. Pretreatment with GRe significantly reduced Bay k-8644-induced p-PKCδ expression in the striatum (FIG. 4).

### Example 5. GRe Significantly Inhibited Bay k-8644-Induced Alterations in Nrf2 Activity and Glutathione Homeostasis

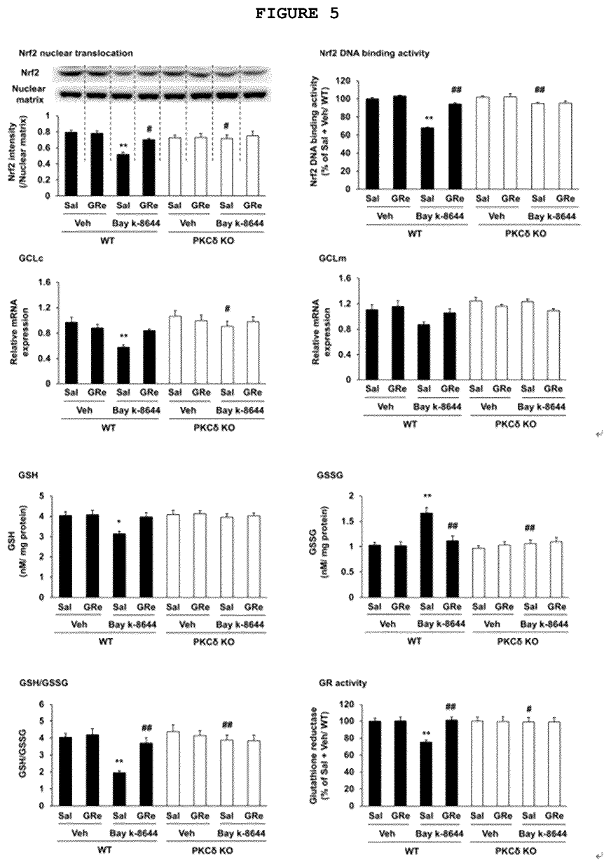

PKCδ signaling has been reported to suppress Nrf2/glutathione pathways in several neurotoxicity models. Because GRe inhibited Bay k-8644-induced p-PKCδ expression, we evaluated whether GRe also affects the associated reductions in Nrf2 activity and alterations in glutathione homeostasis. To further determine whether the inhibitory mechanism of GRe involves PKCδ suppression, PKCδ knockout (KO) mice were included. As Bay k-8644-induced changes in these parameters were most prominent 1 day after administration, the effects of GRe were evaluated at the same time point. In wild-type (WT) mice, Bay k-8644 significantly reduced the nuclear translocation and DNA-binding activity of Nrf2 in the striatum (P<0.01 vs. WT/Saline+Vehicle), and these reductions were significantly attenuated by GRe (Nrf2 nuclear translocation: P<0.05 vs. WT/Saline+Bay k-8644; Nrf2 DNA-binding activity: P<0.01 vs. WT/Saline+Bay k-8644). Bay k-8644-induced reductions in Nrf2 nuclear translocation and DNA-binding activity were also significantly suppressed in PKCδ KO mice (Nrf2 nuclear translocation: P<0.05 vs. WT/Saline+Bay k-8644; Nrf2 DNA-binding activity: P<0.01 vs. WT/Saline+Bay k-8644). However, GRe did not provide additional protective effects in PKCδ KO mice (FIGS. 5A-B). Similarly, Bay k-8644 significantly decreased the mRNA expression of the GCL catalytic subunit (GCLc) in the striatum of WT mice (P<0.01 vs. WT/Saline+Vehicle), and GRe tended to attenuate this reduction. PKCδ gene deletion also significantly suppressed this Bay k-8644-induced reduction (P<0.01 vs. WT/Saline+Bay k-8644), but GRe did not exert additional inhibitory effects in PKCδ KO mice (FIG. 5C). The expression pattern of the GCL modulatory subunit (GCLm) showed a similar trend (FIG. 5D).

Consistently, in the striatum of WT mice, Bay k-8644 significantly decreased GSH levels (P<0.05 vs. WT/Saline+Vehicle), the GSH/GSSG ratio, and GR activity (P<0.01 vs. WT/Saline+Vehicle), while significantly increasing GSSG levels (P<0.01 vs. WT/Saline+Vehicle). Pretreatment with GRe significantly inhibited the Bay k-8644-induced increase in GSSG levels and decrease in the GSH/GSSG ratio in WT mice (P<0.01 vs. WT/Saline+Bay k-8644). PKCδ gene deletion also significantly inhibited the Bay k-8644-induced increase in GSSG levels and decrease in the GSH/GSSG ratio (P<0.01 vs. WT/Saline+Bay k-8644), as well as the reduction in GR activity (P<0.05 vs. WT/Saline+Bay k-8644). However, GRe did not confer additional inhibitory effects in PKCδ KO mice (FIGS. 5E-H). These findings suggest that GRe restores Nrf2 activity and glutathione homeostasis in the striatum of Bay k-8644-treated mice via a PKCδ-dependent mechanism.

### Example 6. GRe Inhibited the Bay k-8644-Induced Reduction in Antioxidant Enzyme Activity in the Striatum

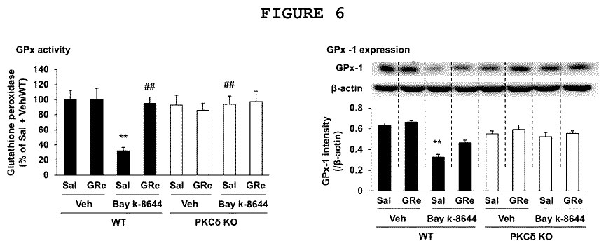

We evaluated the effect of GRe on the Bay k-8644-induced decrease in antioxidant enzyme activity. Because this reduction was most prominent 1 day after Bay k-8644 administration, the effects of GRe were examined at this time point. Bay k-8644 significantly decreased GPx activity in the striatum of WT mice (P<0.01 vs. WT/Saline+Vehicle), and this reduction was significantly inhibited by either GRe (P<0.01 vs. WT/Saline+Bay k-8644) or PKCδ gene deletion (P<0.01 vs. WT/Saline+Bay k-8644). GRe did not exert additional inhibitory effects in PKCδ KO mice (FIGS. 6A-C). GPx expression in the striatum showed a similar pattern (FIG. 6D). These findings indicate that the GRe-mediated enhancement of antioxidant enzyme activity involves the inhibition of PKCδ.

### Example 7. GRe Inhibited Bay k-8644-Induced Self-Injurious Behavior in Mice

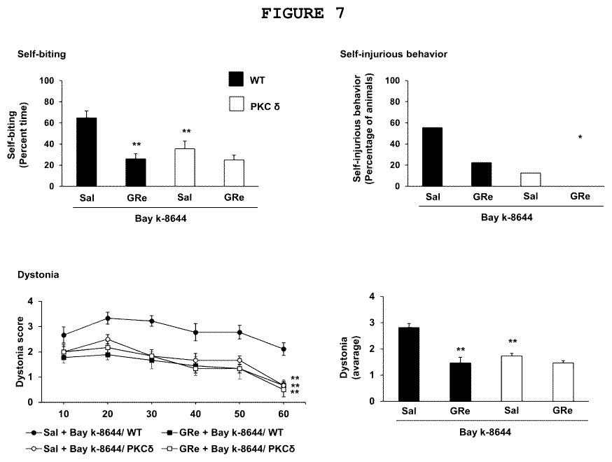

Finally, we evaluated whether GRe suppresses Bay k-8644-induced self-injurious behavior. GRe significantly inhibited self-biting behavior and dystonia in WT mice (P<0.01 vs. WT/Saline+Bay k-8644), and these behaviors were also significantly suppressed in PKCδ KO mice (P<0.01 vs. WT/Saline+Bay k-8644). GRe did not produce additional inhibitory effects in PKCδ KO mice (FIGS. 7A, 7C, 7D). Both GRe pretreatment and PKCδ gene deletion tended to reduce tissue damage caused by self-biting; however, these effects did not reach statistical significance (FIG. 7B). These results indicate that PKCδ contributes to Bay k-8644-induced self-injurious behavior and that GRe attenuates this behavior through the inhibition of PKCδ.

We confirmed that Bay k-8644-induced self-injurious behavior was accompanied by abnormalities in endogenous antioxidant systems and a reduction in Nrf2 activity in the striatum, along with increased PKCδ phosphorylation. Pretreatment with GRe inhibited the Bay k-8644-induced increase in p-PKCδ expression and suppressed the reduction in Nrf2 activity, the downregulation of GCL enzymes, and the decrease in the GSH/GSSG ratio. GRe also significantly inhibited the Bay k-8644-induced decrease in antioxidant enzyme activity and the resulting self-injurious behavior. PKCδ gene deletion similarly suppressed these changes, demonstrating the central role of PKCδ in both Bay k-8644-induced self-injurious behavior and the associated disruption of endogenous antioxidant systems. Furthermore, because GRe did not exert additional inhibitory effects in PKCδ KO mice, these findings indicate that the effects of GRe are mediated through the inhibition of PKCδ. Taken together, these results suggest that GRe mitigates Bay k-8644-induced self-injurious behavior by restoring Nrf2 activity and endogenous antioxidant systems via PKCδ inhibition.

The above detailed description illustrates specific aspects of the present invention. However, such descriptions are merely exemplary embodiments, and it will be apparent to those skilled in the art that the scope of the present invention is not limited by these embodiments. Various modifications, substitutions, and alterations may be made without departing from the spirit and scope of the invention as defined in the appended claims.

What is claimed is:

1. A composition for prevention or treatment of self-injurious behavior comprising ginsenoside Re.

2. The composition according to claim 1, wherein the ginsenoside Re is administered once daily at a dose of 10 to 50 mg/kg.

3. The composition according to claim 1, wherein the self-injurious behavior is non-suicidal self-injury.

4. The composition according to claim 1, wherein the ginsenoside Re alleviates or suppresses self-injurious behavior by inhibiting Protein kinase C δ (PKCδ) phosphorylation, inhibiting changes in glutathione and NRF2 (Nuclear factor erythroid-2-related factor 2) homeostasis, and inhibiting a decrease in antioxidant enzyme activity that appears in association with self-injurious behavior.
